(19) United States
(12) Patent Application Publication
(10) Pub. No.: US 2022/0192471 A1
(43) Pub. Date: Jun. 23, 2022

(54) DETACHABLE SHAFTS FOR ENDOSCOPES

(71) Applicant: 270 SURGICAL LTD., Netanya (IL)

(72) Inventors: Avraham LEVY, Kfar Shmaryahu (IL); Golan SALMAN, Atlit (IL); Amram AIZENFELD, Ramot Menashe (IL)

(21) Appl. No.: 17/611,163

(22) PCT Filed: May 17, 2020
(86) PCT No.: PCT/IL2020/050539

§ 371 Date: Nov. 14, 2021

Related U.S. Application Data
(60) Provisional application No. 62/850,847, filed on May 21, 2019.

(51) Int. Cl. A61B 1/00; A61B 1/05; A61B 1/06

(52) U.S. Cl. CPC A61B 1/00105; A61B 1/00124; A61B 1/00135; A61B 1/05; A61B 1/00103; A61B 1/00101; A61B 1/00177; A61B 1/00128; A61B 1/00181; A61B 1/0625; A61B 1/0676

(57) ABSTRACT

Disclosed is an elongated shaft detachably mountable on a reusable handle of a multi-camera endoscope, The elongated shaft includes a shaft body. The shaft body includes, at a shaft body distal section, at least two cameras and at least one illumination component, and, at a shaft body proximal section, an adaptor. The adaptor is configured to mechanically and electronically detachably couple to a coupling interface on a distal section of the handle, such as to mount the elongated shaft on the handle. The adaptor is further configured to dictate a preferred mounting orientation such that the at least two cameras provide a combined and predetermined at least about 270 degrees horizontal field-of-view (FOV) of a target area within an anatomical cavity when the elongated shaft is mounted on the handle.

### TECHNICAL FIELD

The present disclosure relates generally to rigid and semi-rigid endoscopes.

### BACKGROUND

An endoscope is a medical device used to image an anatomical site (e.g. a body cavity, a hollow organ). Unlike some other medical imaging devices, the endoscope is inserted into the anatomical site (e.g. through small incisions made on the skin of the patient). An endoscope can be employed not only to inspect an anatomical site and e.g. organs therein (and diagnose a medical condition in the anatomical site) but also as a visual aid in surgical procedures. Medical procedures involving endoscopy include laparoscopy, arthroscopy, cystoscopy, ureteroscopy, and hysterectomy.

### SUMMARY

Aspects of the disclosure, according to some embodiments thereof, relate to elongated shafts of rigid and semi-rigid endoscopes. More specifically, but not exclusively, aspects of the disclosure, according to some embodiments thereof, relate to detachable, elongated shafts—of rigid and semi-rigid endoscopes—including a sterile sleeve wrapped on the elongated shaft. The sleeve is configured to be pulled over the handle (when the shaft is mounted on the handle), such as to fully cover the handle. The sleeve is configured (in its pulled over state) to prevent body fluids (e.g. blood) and debris (e.g. tissue) from reaching the handle, thereby obviating the necessity of sterilizing the handle following each usage of the endoscope. The handle may further be configured to (i) afford a user (e.g. a surgeon) a comfortable and secure grip of the handle, and/or (ii) to be manipulated by a robotic arm or robotic gripping means (e.g. controlled by the surgeon).

According to some embodiments, the shaft is disposable (while the handle is reusable), thereby obviating the necessity of sterilizing the shaft following each usage of the endoscope. Advantageously, this may allow for reduction of manufacturing costs, as the shaft is no longer required to withstand e.g. autoclave sterilization.

Advantageously, according to some embodiments, wherein the endoscope includes a plurality of cameras positioned at a distal section of the shaft, the shaft may be mounted on the handle only at a preferred mounting orientation, thereby ensuring that a combined and consistent panoramic view is obtained from the cameras.

Thus, according to an aspect of some embodiments, there is provided an elongated shaft detachably mountable on a reusable handle of a multi-camera endoscope. The elongated shaft includes a shaft body. The shaft body includes at a shaft body distal section at least two cameras and at least one illumination component, and, at a shaft body proximal section, an adaptor. The adaptor is configured to mechanically and electronically detachably couple to a coupling interface on a distal section of the handle, such as to mount the elongated shaft on the handle. The adaptor is further configured to dictate a preferred mounting orientation such that the at least two cameras provide a combined and predetermined at least about 270 degrees horizontal field-of-view (FOV) of a target area within an anatomical cavity when the elongated shaft is mounted on the handle.

According to some embodiments, the elongated shaft further includes a sterile sleeve wrapped on the shaft body. The sleeve includes a sleeve first end, which is circumferentially attached to the shaft body proximal section, and a sleeve second end. The sleeve is configured to be proximally pulled over the handle when the elongated shaft is mounted on the handle, thereby unwrapping the sleeve. The attachment between the sleeve first end and the shaft body proximal section is fluid-tight, thereby preventing fluids and debris from anatomical cavities, into which the elongated shaft is insertable, from reaching the handle when the sleeve is pulled over the handle.

According to some embodiments, the at least two cameras include a front camera on a distal tip of the shaft and a first side-camera.

According to some embodiments, the at least two cameras further include a second side-camera. The first side-camera and the second side-camera are positioned on opposite sides of the shaft. The first side-camera is positioned distally relative to the second side-camera.

According to some embodiments, the elongated shaft is disposable.

According to some embodiments, the shaft body distal section is detachable.

According to some embodiments, the shaft body distal section is reusable and the rest of the of the elongated shaft is disposable.

According to some embodiments, the adaptor includes an interlocking component, which is complementary to an interlocking component on the coupling interface. The interlocking components are configured to prevent coupling between the adaptor and the coupling interface except at the preferred orientation.

According to some embodiments, the adaptor includes a keyed component, which is complementary to a keyed component on the coupling interface. The keyed components are configured to prevent coupling between the adaptor and the coupling interface except at the preferred orientation.

According to some embodiments, the interlocking component includes one or more snap-female receptors. Each of the snap-female receptors is configured to snap-engage a corresponding pin on the coupling interface.

According to some embodiments, the interlocking component includes one or more pins. Each of the pins is configured to be snap-engaged by corresponding snap-female receptor in the coupling interface.

According to some embodiments, the adaptor includes a spring-loaded pin component, which is configured to engage a corresponding flat conductive surface on the coupling interface when the adaptor is coupled to the coupling interface at the preferred orientation.

According to some embodiments, the adaptor includes a flat conductive surface, which is configured to engage a spring-loaded pin component on the coupling interface when the adaptor is coupled to the coupling interface at the preferred orientation.

According to some embodiments, the shaft body is rigid or semi-rigid.

According to some embodiments, each of the at least one illumination component is or includes discrete light source.

According to an aspect of some embodiments, there is provided a multi-camera endoscope including an elongated shaft as described above, and a reusable handle. The elongated shaft is detachably mountable on the handle.

According to some embodiments, the elongated shaft and/or the handle include an authentication mechanism configured to prevent operation of the multi-camera endoscope unless the shaft is authenticated.

According to some embodiments, a used shaft is inauthentic.

According to an aspect of some embodiments, there is provided a detachable elongated shaft mountable on a reusable handle of an endoscope. The elongated shaft includes:
At least one camera and at least one illumination component located at a shaft distal section.
An adaptor located at a shaft proximal section.
A sterile sleeve wrapped on the elongated shaft.

The sterile sleeve includes a sleeve first end, which is circumferentially attached to the shaft proximal section, and a sleeve second end. The adaptor is configured to mechanically and electronically detachably couple to a coupling interface on a distal section of the reusable handle, such as to mount the elongated shaft on the handle. The sterile sleeve is configured to be proximally pulled over the handle when the elongated shaft is mounted on the handle, thereby unwrapping the sterile sleeve. The attachment between the sleeve first end and the shaft proximal section is fluid-tight, thereby preventing fluids and debris from anatomical cavities, into which the elongated shaft is insertable, from reaching the handle when the sleeve is pulled over the handle.

According to some embodiments, in an initial configuration, the sleeve second end is positioned distally to the sleeve first end. When the sterile sleeve has been pulled over the handle, the sleeve second end is positioned proximally to the sleeve first end and a first surface of the sterile sleeve and a second surface of the sterile sleeve have been inverted.

According to some embodiments, the sterile sleeve is tapered such that a circumference of the sleeve second end is greater than a circumference of a sleeve intermediate segment.

According to some embodiments, the sleeve first end is sealably attached to the shaft proximal section by a glue, a band, a snap connector, and/or a tape, and/or is heat-fused or ultrasonically welded thereto.

According to some embodiments, the elongated shaft is disposable.

According to some embodiments, the shaft distal section is detachable.

According to some embodiments, the shaft distal section is reusable and the rest of the elongated shaft is disposable.

According to some embodiments, each of the at least one illumination component is or includes a discrete light source.

According to some embodiments, the elongated shaft is rigid or semi-rigid.

According to some embodiments, the sterile sleeve is further configured to be pulled over a utility cable attached to the handle when the elongated shaft is mounted on the handle.

According to some embodiments, the sterile sleeve is further configured to be pulled over a connector of an external control unit, to which the utility cable is configured to be connected, when the elongated shaft is mounted on the handle and the utility cable is connected to the connector.

According to an aspect of some embodiments, there is provided a multi-camera endoscope including an elongated shaft (with a sterile sleeve wrapped thereon) as described above, and a reusable handle. The elongated shaft is mounted on the handle.

According to an aspect of some embodiments, there is provided an imaging component detachably mountable on a distal end of an elongated member of a multi-camera endoscope (such that the imaging component and elongated member form the shaft of the endoscope). The imaging component includes at least two cameras, at least one illumination component, and an adaptor electrically associated with the at least two cameras and the at least one illumination component. The adaptor is configured to mechanically and electronically detachably couple the imaging component to the distal end of the elongated member. The adaptor is further configured to dictate a preferred coupling orientation, such that the at least two cameras provide a combined and predetermined horizontal field-of-view (FOV) of at least about 270 degrees of a target area within an anatomical cavity, when coupled to the elongated member.

According to some embodiments, the imaging component is disposable.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIGS. 1A and 1B are schematic, perspective views of a rigid endoscope including a reusable handle and a detachable elongated shaft, the endoscope is depicted in an assembled state and disassembled state, respectively, according to some embodiments;

FIG. 2 schematically depicts a medical imaging system including the rigid endoscope of FIG. 1A, according to some embodiments;

FIG. 3 schematically depicts the elongated shaft of FIG. 1A and a field-of-view provided by cameras positioned in a distal section of the elongated shaft, according to some embodiments;

FIG. 4 is a schematic, perspective view of specific embodiments of the elongated shaft rigid endoscope of FIG. 1A, wherein the distal section of the elongated shaft is detachable;

FIGS. 5A and 5B are schematic, perspective views of specific embodiments of the rigid endoscope of FIG. 1A, depicting a coupling mechanism between the elongated shaft and the handle;

FIGS. 6A and 6B are schematic, perspective views of specific embodiments of the rigid endoscope of FIG. 1A, depicting a coupling mechanism between the elongated shaft and the handle;

FIGS. 7A and 7B are schematic, perspective views of specific embodiments of the rigid endoscope of FIG. 1A, depicting a coupling mechanism between the elongated shaft and the handle;

FIG. 8 is a schematic, perspective view of a rigid endoscope including a reusable handle and a detachable elongated shaft, the elongated shaft including a sterile sleeve wrapped thereon, according to some embodiments;

FIGS. 9A-9D schematically depict successive stages in the pulling of the sleeve of FIG. 8 over the handle of FIG. 8, thereby unwrapping the sleeve, according to some embodiments;

FIGS. 10A and 10B schematically depict successive stages in the removal of the sleeve of FIG. 8 from the handle of FIG. 8, following the pulling of the sleeve over the handle, according to some embodiments; and

FIG. 11 is a schematic, perspective view of a rigid endoscope including a reusable handle and a detachable elongated shaft, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. In such embodiments, for example, the statement “the length of the element is equal to about 1 millimeter” is equivalent to the statement “the length of the element is between 0.99 millimeters and 1.01 millimeters”.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol ⊙ may be used to represent an axis pointing “out of the page”, while the symbol ⊗ may be used to represent an axis pointing “into the page”.

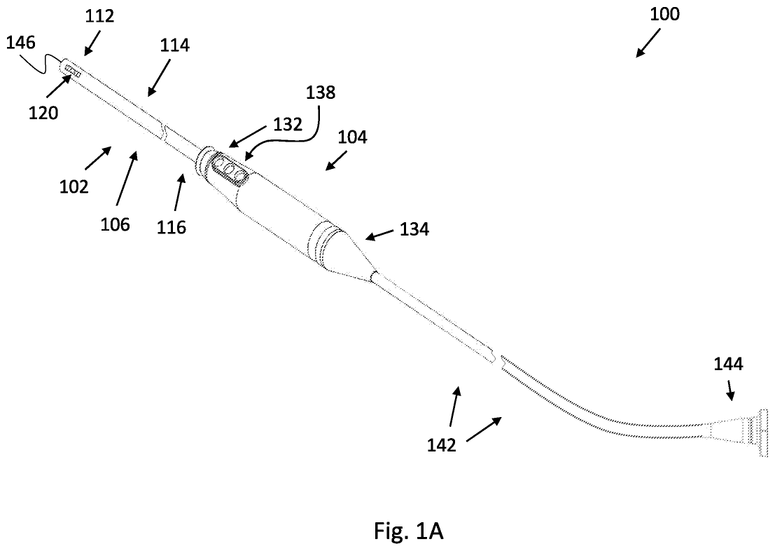

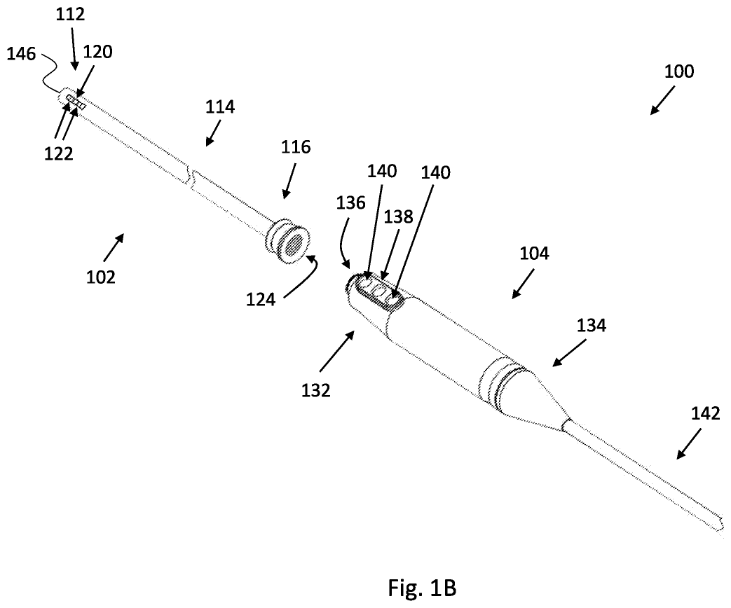

FIGS. 1A and 1B schematically depict a rigid endoscope 100, according to some embodiments. Endoscope 100 includes an elongated shaft 102, configured to be inserted into an anatomical site (e.g. an anatomical cavity), and a handle 104, configured to be held by a user (e.g. a surgeon) of endoscope 100 and to facilitate guiding and manipulation of shaft 102 (particularly a distal section thereof) within the anatomical site. Shaft 102 is configured to be detachably mountable on handle 104. FIG. 1A shows endoscope 100 in an assembled state, wherein shaft 102 is mounted on handle 104. FIG. 1B shows endoscope 100 in a disassembled state, wherein shaft 102 is detached from handle 104.

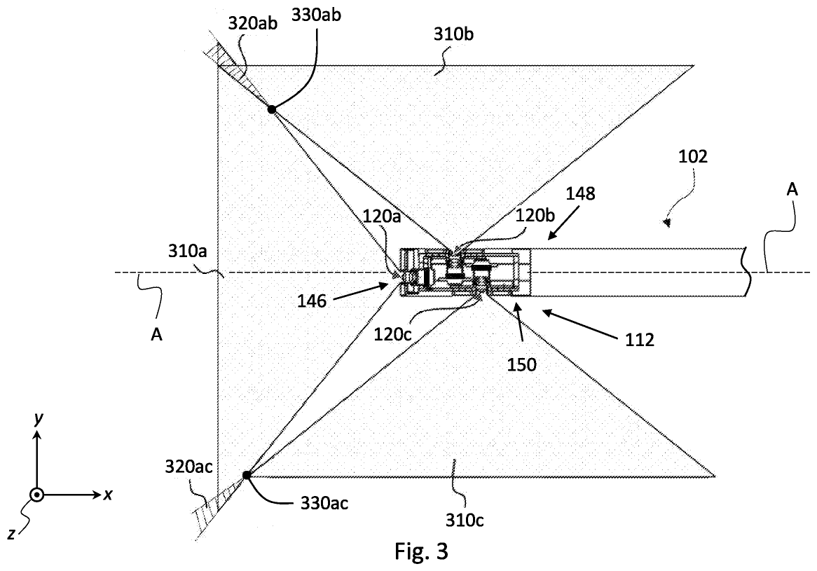

Shaft 102 includes a shaft body 106, e.g. a rigid tubular member. Shaft 102 includes a shaft distal section 112, a shaft central section 114, and a shaft proximal section 116 (i.e. a distal section, a central section, and a proximal section, respectively, of shaft 102). Shaft distal section 112 includes at least two cameras 120 (e.g. a front camera, as seen for example in FIG. 3, and at least one side camera) and illumination components 122 (the illumination components of one of the side cameras are numbered in FIG. 1B), such as light emitting diodes (LEDs). Shaft proximal section 116 includes an adaptor 124, the function thereof is described below.

According to some embodiments, each of illumination components 122 is or includes a discrete light source. According to some embodiments, illumination components 122 may be mounted on one or more PCBs in shaft distal section 112. According to some embodiments, wherein illumination components 122 include LEDs, the LEDs may include, for example, one or more white light LEDs, infrared LEDs, a near infrared LEDs, an ultraviolet LED, and/or a combination thereof. It is noted that in embodiments wherein illumination components include LEDs configured to produce light outside the visible spectrum (e.g. an infrared LED), cameras 120 will include sensors configured to detect such light (e.g. infrared light). That is, cameras 120 will have capacities of e.g. infrared cameras and so on.

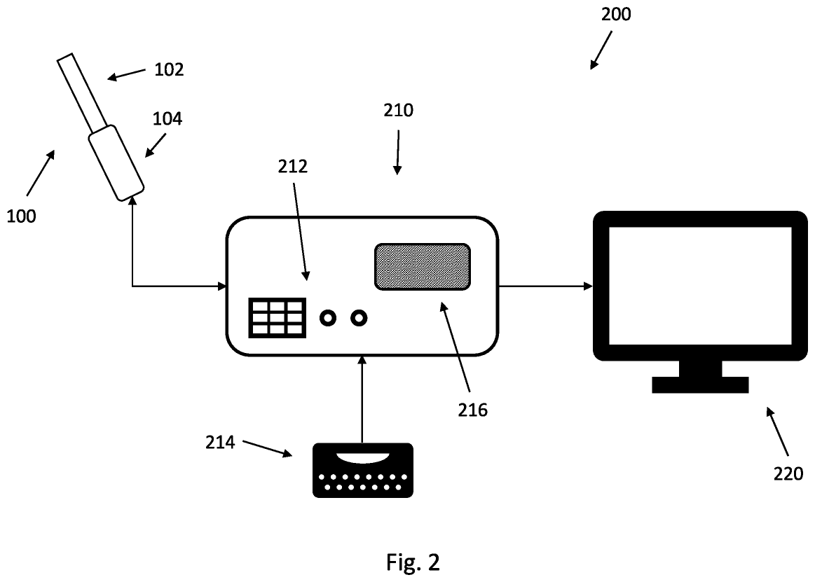

According to some embodiments, illumination components 122 include the distal tips of respective optical fibers (not shown). According to some such embodiments, handle 104 may include one or more light sources connected to one or more optical fibers extending through handle 104 and shaft 102. The optical fibers are configured to guide the light produced by the light sources from handle 104 to shaft distal section 112, wherefrom the guided light may be shone such as to illuminate the field-of-view of cameras 120. According to some embodiments, the light sources may be external to handle 104, being positioned, for example, in a main control unit such as the main control unit depicted in FIG. 2.

Handle 104 includes a handle distal section 132 and a handle proximal section 134 (i.e. a distal section and a proximal section of handle 104, respectively). Handle distal section 132 may include a coupling interface 136 configured to be mechanically coupled and electronically coupled with adaptor 124, such as to assemble endoscope 100 (i.e. mount shaft 102 on handle 104) and to functionally associate cameras 120, and optionally illumination components 122, with electronic circuitry/components in handle 104 (and thereby with external systems to which handle 104 is configured to be connected, as depicted in FIG. 2). According to some embodiments, adaptor 124 may be “male”, or include male components, and coupling interface 136 may be “female”, or include corresponding female components. According to some embodiments, coupling interface 136 may be male, or include male components, and adaptor 124 may be female, or include corresponding female components. According to some embodiments, adaptor 124 may include pins and coupling interface 136 may include matching pinholes configured for electronically coupling adaptor 124 and coupling interface 136. According to some embodiments, coupling interface 136 may include pins and adaptor 124 may include matching pinholes configured for electronically coupling adaptor 124 and coupling interface 136. According to some embodiments, adaptor 124 may include spring-loaded pins and coupling interface 136 may include flat, or substantially flat, conductive surfaces (or matching pinholes) configured for electronically coupling adaptor 124 and coupling interface 136. According to some embodiments, coupling interface 136 may include spring-loaded pins and adaptor 124 may include flat, or substantially flat, conductive surfaces (or matching pinholes) configured for electronically coupling adaptor 124 and coupling interface 136.

According to some embodiments, wherein illumination components 122 include the distal tips of optical fibers and handle 104 includes one or more optical fibers for guiding the light from a light source therein or external thereto to shaft distal section 112, coupling interface 136 and adaptor 124 will further include an optical interface (not shown) for connecting the optical fiber(s) in handle 104 to the optical fiber(s) in shaft 102.

Handle distal section 132 may include a user control interface 138 configured to allow a user to control endoscope 100 functions. User control interface 138 may be functionally associated with cameras 120 and illumination components 122 via the electronic coupling between shaft 102 and handle 104 which is provided by adaptor 124 and coupling interface 136. According to some embodiments, user control interface 138 may allow, for example, to control zoom, focus, record/stop recording, and/or freeze frame functions of cameras 120 and/or to adjust the light intensity provided by illumination components 122. According to some embodiments, user control interface 138 may allow to control the presentation of video streams from cameras 120 on an associated monitor (such as the monitor depicted in FIG. 2). For example, according to some embodiments, user control interface 138 may allow to present the video streams side-by-side, and/or with a video stream from one of the cameras (e.g. a front camera) being displayed larger than the video streams from the other cameras (e.g. side cameras), and/or to display two copies of one of the video streams and allow to manipulate one of the copies. User control interface 138 may include one or more buttons 140 (as a non-limiting example, three buttons, as depicted in FIGS. 1A and 1B; not all of which are numbered), knobs, switches, a touch panel, and/or the like.

Each of cameras 120 may include a sensor, such as a charge coupled device (CCD) image sensor or a complementary metal oxide semiconductor (CMOS) image sensor, and a camera lens (e.g. an extreme wide-angle lens) or a lens assembly. According to some embodiments, each of the sensors may be mounted on a respective printed circuit board (PCB). According to some embodiments, all the sensors may be mounted on a common PCB. Cameras 120 may be configured to provide a continuous/panoramic field-of-view (FOV), as elaborated on below in the description of FIG. 3.

According to some embodiments, shaft 102 and/or handle 104 include an authentication mechanism(s) configured to prevent operation of endoscope 100 unless shaft 102 is authenticated. According to some such embodiments, a “used” shaft 102, such as a shaft that has already been used in an endoscopy procedure, is inauthentic. According to some embodiments, shaft 102 and/or handle 104 may include processing circuitry configured to identify whether a shaft is authentic. The processing circuitry may be configured to prevent the electronic coupling of shaft 102 to handle 104 unless shaft 102 is authenticated. For example, handle 104 may include an electrical switch which, when open (and when shaft 102 is mounted handle 104), electrically decouples shaft 102 from handle 104, with the processing circuitry being configured to close the electrical switch only when the shaft is authenticated.

FIG. 2 schematically depicts a medical imaging system 200, according to some embodiments. Medical imaging system 200 includes endoscope 100, a main control unit 210, and a monitor 220. According to the convention adopted herein, a same reference numeral in different figures refers to the same object (e.g. device, element). Thus, for example, in FIGS. 1A, 1B, and 2, the reference numeral 100 refers to the same endoscope (i.e. endoscope 100). Similarly, in FIGS. 1A, 1B, 2 and 3, the reference numeral 102 refers to the same shaft (i.e. shaft 102 of endoscope 100).

Endoscope 100 and monitor 220 may each be functionally associated with main control unit 210. Main control unit 210 includes processing circuitry (e.g. one or more processors and memory components) configured to process (digital data) from cameras 120 (not shown in FIG. 2 but depicted in FIGS. 1A and 1B), such as to display the captured images and video on monitor 220. In particular, the processing circuitry may be configured to process the digital data received from each of cameras 120, such as to produce therefrom video files/streams providing a panoramic view of the anatomical site, as explained below in the description of FIG. 3. According to some embodiments, the processing circuitry may be configured to process the data received from cameras 120 to produce a combined video stream providing a continuous and consistent (seamless) panoramic view of the anatomical site.

Main control unit 210 may include a user interface 212 (e.g. buttons and/or knobs, a touch panel, a touch screen) configured to allow a user to operate main control unit 210 and/or may allow control thereof using one or more input devices 214, e.g. an external user control interface connectable thereto such as a keyboard, a mouse, a portable computer, and/or even a mobile computational device e.g. a smartphone or a tablet. According to some embodiments, input devices 214 may include a voice controller. According to some embodiments, main control unit 210 may further be configured to partially or even fully operate cameras 120 and illumination components 122 (shown in FIGS. 1A and 1B). Some operational aspects may be operated automatically, for example, according to some embodiments, the supply of power to endoscope 100 components, such as cameras 120 and illumination components 122, while other operational aspects or functions may be operated using user interface 212 and/or input devices 214. According to some embodiments, main control unit 210 may include a display 216 (for example, the touch screen and/or another screen) for presenting information regarding the operation of endoscope 100, such as the brightness levels of cameras 120, zoom options, focus, and the like. According to some embodiments, wherein display 216 is a touch screen, display 216 may further allow controlling for example, the zoom, focus, record/stop recording functions, freeze frame function, and/or the brightness of cameras 120, and/or to adjust the light intensity of illumination components 122. According to some embodiments, the choice of information presented may be controlled using user interface 212, user control interface 138, and/or input devices 214.

According to some embodiments, endoscope 100 is functionally associated with main control unit 210 via a utility cable 142 (shown in FIGS. 1A and 1B) connected to or configured to be connected to handle proximal section 134, and further configured to be connected to main control unit 210 (via, for example, a plug 144 or a port). Cable 142 may include at least one data cable for receiving video signals from cameras 120, and at least one power cable for providing electrical power to cameras 120 and to illumination components 122, as well as to operationally control parameters of cameras 120 and illumination components 122, such as the light intensity. Additionally or alternatively, according to some embodiments, endoscope 100 may include a wireless communication unit (e.g. a Bluetooth antenna) configured to communicatively associate endoscope 100 with main control unit 210. According to some embodiments, endoscope 100 is configured to be powered by a replaceable and/or rechargeable battery included therein, i.e. inside handle 104. According to some embodiments, wherein illumination components 122 include the distal tips of optical fibers and wherein the light source(s) is positioned in main control unit 210, cable 142 will also include one or more optical fibers configured to guide the light produced by the light source(s) to an optical fiber(s) in handle 104, wherefrom the light will be guided to optical fibers in shaft 102.

Monitor 220 is configured to display images and, in particular, to stream video captured by cameras 120, and may be connected to main control unit 210 by a cable (e.g. a video cable) or wirelessly. According to some embodiments, monitor 220 may be configured to display thereon information regarding the operation of endoscope 100, as specified above. According to some embodiments, monitor 220, or a part thereof, may function as a touch screen. According to some such embodiments, the touch screen may be used to operate main control unit 210. According to some embodiments, images/videos from different cameras (from cameras 120) may be displayed separately (e.g. side-by-side, in an equal aspect ratio, in multiple copies of one or more of the video streams, and the like) on monitor 220, and/or may be presented as a single panoramic image/video. According to some embodiments, user interface 212 and/or input devices 214 are configured to allow switching between images/videos corresponding to different FOVs (of different cameras). For example, according to some embodiments, wherein cameras 120 include a front camera 120a, a first side-camera 120b, and a second side-camera 120c: switching between footage captured by front camera 120a to footage captured by first side camera 120b, switching between footage captured by front camera 120a to footage captured by second side-camera 120c, or switching between a panoramic video generated from the footage of all of cameras 120a, 120b, and 120c to footage captured by one of cameras 120a, 120b, or 120c. Cameras 120a, 120b, and 120c are depicted together in FIG. 3. According to some embodiments, main control unit 210 may be associated with a plurality of monitors, such as monitor 220, thereby allowing to display different videos and images on each. For example, main control unit 210 may be associated with four monitors, such as to allow displaying videos from each of cameras 120a, 120b, 120c on three of the monitors, respectively, and a panoramic video (corresponding to the combination of the three videos) on the fourth monitor, which may be wider than the other three.

The field-of-view (FOV) provided by endoscope 100 is the combination of the respective FOVs provided by each of cameras 120. Cameras 120 may be configured to provide a continuous and consistent FOV, or at least a continuous and consistent horizontal FOV (HFOV), as explained below. FIG. 3 schematically depicts shaft distal section 112 (of shaft 102) and a combined HFOV provided by front camera 120a, first side-camera 120b, and second side-camera 120c, according to some embodiments. Front camera 120a is positioned within shaft distal section 112 on a front surface 146 of the distal tip (not numbered) of shaft distal section 112, with a lens assembly (not numbered) of front camera 120a being exposed on front surface 146. First side-camera 120b is positioned within shaft distal section 112 on a first side-surface 148 thereof, with a lens assembly (not numbered) of first side-camera 120b being exposed on first side-surface 148. Second side-camera 120c is positioned within shaft distal section 112 on a second side-surface 150 thereof, with a lens assembly (not numbered) of second side-camera 120c being exposed on second side-surface 150. First side-surface 148 is opposite to second side-surface 150. According to some embodiments, first side-camera 120b and second side-camera 120c are not positioned back-to-back. According to some embodiments, the distance between the center-point of first side-camera 120b (i.e. the center of a lens of first side-camera 120b) and front surface 146 is between about 5 millimeters to about 20 millimeters and the distance between the center-point of first side-camera 120b and the center-point of second side-camera 120c may be up to about 10 millimeters.

The combined HFOV is formed by a front HFOV 310a, a first side-HFOV 310b, and a second side-HFOV 310c of front camera 120a, first side-camera 120b, and second side-camera 120c, respectively. Each of HFOVs 310a, 310b, and 310c lies on the xy-plane. HFOV 310a is positioned between HFOVs 310b and 310c and overlaps with each. A first overlap area 320ab corresponds to an area whereon HFOVs 310a and 310b overlap. In other words, first overlap area 320ab is defined by the intersection of the xy-plane with the overlap region (volume) of the FOVs of front camera 120a and first side-camera 120b. Similarly, a second overlap area 320ac corresponds to an area whereon HFOVs 310a and 310c overlap. A first intersection point 330ab is defined as the point in first overlap area 320ab which is closest to front camera 120a. It is noted that first intersection point 330ab also corresponds to the point in first overlap area 320ab which is closest to first side-camera 120b. Similarly, a second intersection point 330ac is defined as the point in second overlap area 320ac which is closest to front camera 120a. It is noted that second intersection point 330ac also corresponds to the point in second overlap area 320ac which is closest to second side-camera 120c.

The combined FOV (of cameras 120a, 120b, and 120c) is continuous since the panoramic view provided thereby does not contain any gaps (as would have been the case had HFOV 310a not overlapped with at least one of HFOVs 310b and 310c). Further, the combined HFOV is consistent (i.e. seamless) in the sense that the magnifications of the lenses of each of cameras 120a, 120b, and 120c are compatible such that the view of objects (e.g. organs or surgical tools), or parts of objects, in the overlap areas are not distorted and the (overall) combined HFOV merges the combined HFOVs of each front HFOV 310a and first side-HFOV 310b, and front HFOV 310a and second side-HFOV 310c, in a seamless manner Thus, the magnification provided by the lens of first side-camera 120b may be slightly larger than the magnification provided by the lens of front camera 120a to compensate for first intersection point 330ab being closer to front camera 120a than to first side-camera 120b. That is, D₁<D₂, wherein D₁ is the distance between front camera 120a and first intersection point 330ab, and D₂ is the distance between first intersection point 330ab and first side-camera 120b. Similarly, the magnification provided by the lens of second side-camera 120c may be slightly larger than the magnification provided by the lens of front camera 120a to compensate for second intersection point 330ac being closer to front camera 120a than to second side-camera 120c.

According to some embodiments, the combined HFOV spans between about 220 degrees to about 270 degrees, between about 240 degrees to about 300 degrees, or between about 240 degrees to about 340 degrees. Each possibility corresponds to separate embodiments. According to some embodiments, the combined HFOV spans at least about 270 degrees. According to some embodiments, for example, each of HFOVs 310a, 310b, and 310c may measure between about 85 degrees to about 120 degrees, between about 90 degrees to about 110 degrees, or between about 95 degrees to about 120 degrees. Each possibility corresponds to separate embodiments.

According to some embodiments, shaft 102 may measure between about 100 millimeters and about 500 millimeters in length, and shaft body 106 may have a diameter measuring between about 2.5 millimeters and about 15 millimeters. According to some embodiments, front camera 120a may be offset relative to a longitudinal axis A, which centrally extends along the length of shaft 102. According to some embodiments, the distance between second side-camera 120c and front surface 146 is greater than the distance between first side-camera 120b and front surface 146.

According to some embodiments, front camera 120a may be offset relative to the longitudinal axis A by up to about 0.05 millimeters, up to about 0.1 millimeters, up to about 0.5 millimeters, up to about 1.0 millimeters, up to about 1.5 millimeters, up to about 5.0 millimeters, or up to about 7.0 millimeters. Each possibility corresponds to separate embodiments. According to some embodiments, for example, front camera 120a may be offset relative to the longitudinal axis A by between about 0.05 millimeters to about 0.1 millimeters, about 0.5 millimeters to about 1.5 millimeters, about 1.0 millimeter to about 5.0 millimeters, about 1.5 millimeters to about 5.0 millimeters, or about 1.0 millimeters to about 7.0 millimeters. Each possibility corresponds to separate embodiments. According to some embodiments, first side-camera 120b may be positioned at a distance of up to about 1.0 millimeters, up to about 5.0 millimeters, or up to about 15.0 millimeters from front surface 146. Each possibility corresponds to separate embodiments. According to some embodiments, second side-camera 120c may be positioned at a distance of up to about 1.0 millimeters, up to about 5.0 millimeters, up to about 15.0 millimeters, or up to about 25.0 millimeters from front surface 146, such as to optionally be positioned farther from front surface 146 than first-side-camera 120b. Each possibility corresponds to separate embodiments. According to some embodiments, for example, first side-camera 120b may be positioned at a distance of between about 1.0 millimeters to about 5.0 millimeters or about 5.0 millimeters to about 15.0 millimeters from front surface 146. Each possibility corresponds to separate embodiments. According to some embodiments, second side-camera 120c may be positioned at a distance of between about 1.0 millimeters to about 5.0 millimeters, about 5.0 millimeters to about 15.0 millimeters, or about 5.0 millimeters to about 25.0 millimeters from front surface 146, such as to optionally be positioned farther from front surface 146 than first-side-camera 120b. Each possibility corresponds to separate embodiments. According to some embodiments, the positioning of cameras 120 on shaft distal section 112 is selected such as to minimize the space occupied by cameras 120 and reduce the diameter of shaft distal section 112, while affording a continuous and consistent HFOV of at least about 270 degrees.

According to some embodiments, each of cameras 120 is associated with a respective illumination component from illumination components 122, which is configured to illuminate the FOV of the camera. Thus, according to some embodiments, front camera 120a may be associated with a respective front illumination component (not shown in the figures), first side-camera 120b may be associated with a respective first side-illumination component, and second side-camera 120c may be associated with a respective second side-illumination component.

According to some embodiments, not depicted in the figures, cameras 120 include only two cameras, both of which are side cameras with fish eye lenses. In such embodiments, shaft distal section 112 may taper in the distal section, such that the cameras provide a continuous HFOV. According to some embodiments, not depicted in the figures, cameras 120 include only two cameras: a front camera and a side camera.

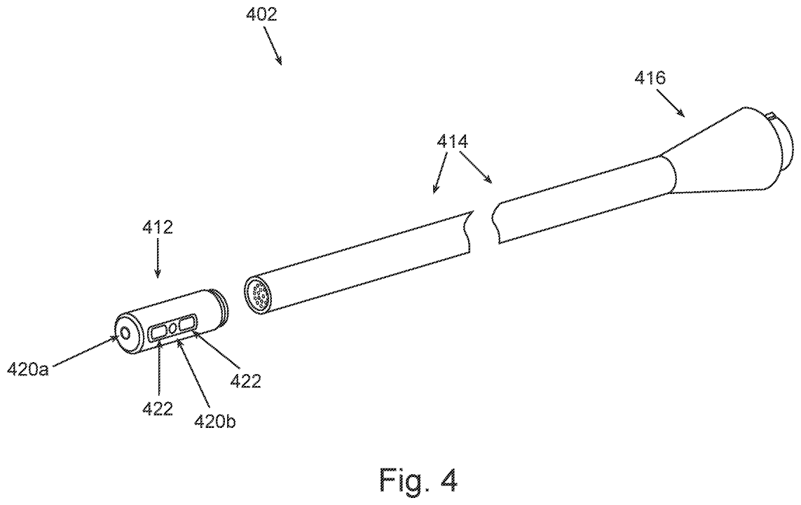

FIG. 4 schematically depicts an elongated shaft 402, which is a specific embodiment of elongated shaft 102 (and can therefore be mounted on handle 104). Shaft 402 includes a shaft distal section 412, a shaft central section 414, and a shaft proximal section 416. Shaft 402 is characterized by shaft distal section 412 being detachable from the rest of shaft 402 (i.e. shaft central section 414 and shaft proximal section 416). That is, shaft distal section 412 may be detached from shaft central section 414. According to some embodiments, shaft central section 414 and shaft proximal section 416—which may be detachable or non-detachable from one another—are disposable, while shaft distal section 412 is reusable and is configured to be repeatedly sterilized, e.g. following each use of the endoscope (i.e. an endoscope including shaft 402 and handle 104). Thus, optical components (i.e. the cameras) need not be discarded after each use. According to some other embodiments, shaft central section 414 and shaft proximal section 416—which may be detachable from one another or non-detachable from one another—are made of reusable materials/components such as to conform with sterilization requirements, while shaft distal section 412 is made of disposable materials/components and may be discarded after a single use.

Also indicated in FIG. 4 are a front camera 420a, a first side-camera 420b, and illumination components 422, which are specific embodiments of front camera 120a, first side-camera 120b, and illumination components 122. A second side-camera, which is a specific embodiment of second side-camera 120c, and illumination components associated therewith, are not visible in FIG. 4.

Making reference again to FIG. 1B, according to some embodiments, adaptor 124 and coupling interface 136 are configured to be coupled at a preferred orientation there between, such that cameras 120 provide a combined HFOV of at least about 270 degrees. That is, in the preferred orientation each of the spring-loaded pins engages an intended target flat conductive surface, so that a consistent FOV is obtained. According to some embodiments, adaptor 124 and coupling interface 136 are configured to be interlocked at the preferred orientation. Non-limiting examples of such coupling and interlocking mechanisms are described below. According to some embodiments, adaptor 124 and/or coupling interface 136 are further configured to dictate the preferred (mounting and coupling) orientation. The preferred orientation ensures that a spring-loaded pin, conveying data from e.g. front camera 120a, engages a flat-conductive surface configured to relay front camera 120a data. In particular, situations wherein a spring-loaded pin, conveying data from e.g. front camera 120a, engages a flat-conductive surface configured to relay e.g. first side camera 120b data, are thereby avoided.

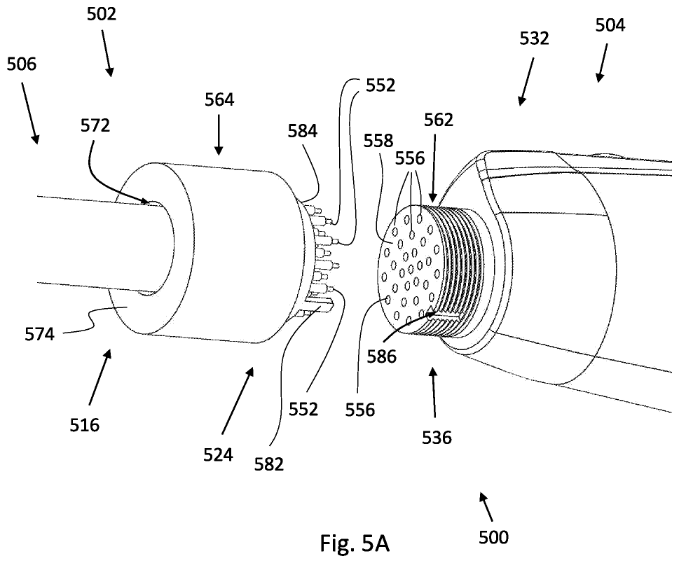

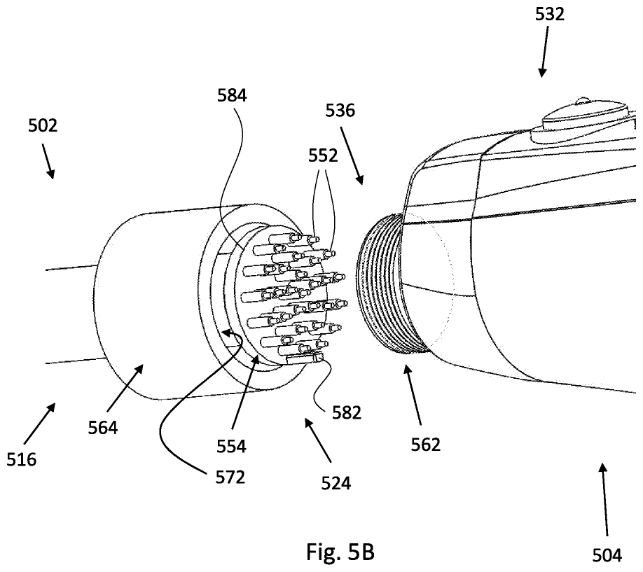

FIGS. 5A and 5B are schematic, perspective (partial) views of a rigid endoscope 500, according to some embodiments. Endoscope 500 is a specific embodiment of endoscope 100 and includes an elongated shaft 502 and a handle 504, which are specific embodiments of elongated shaft 102 (or elongated shaft 402) and handle 104, respectively. More specifically, in FIGS. 5A and 5B a shaft proximal section 516 and a handle distal section 532 are depicted. Shaft proximal section 516 includes an adaptor 524 and handle distal section 532 includes a coupling interface 536. Adaptor 524 and coupling interface 536 are specific embodiments of adaptor 124 and coupling interface 136 and are configured to be interlocked (mechanically coupled) and electronically coupled via a threaded and optionally keyed interlocking mechanism, as described below.

According to some embodiments, and as depicted in FIGS. 5A and 5B, adaptor 524 includes one or more spring-loaded pins 552 (not all of which are numbered) proximally projecting from a shaft proximal tip member 554 (numbered in FIG. 5B), and coupling interface 536 includes one or more corresponding flat conductive surfaces 556 (not all of which are numbered) on a handle distal end 558. Nevertheless, it will be understood that other electronic coupling mechanisms are applicable, such as (non-spring loaded) pins and corresponding pinholes (as described hereinabove in the description of FIGS. 1A and 1B). Coupling interface 536 may include a threaded circumferential surface 562. Shaft proximal tip member 554 may be characterized by a greater diameter than the rest of a shaft body 506 of shaft 502. Adaptor 524 may include a threaded cover 564 (i.e. threaded on an inner surface thereof). Threaded cover 564 may be mounted on shaft body 506 (i.e. the body of shaft 502), distally to shaft proximal tip member 554, and may be configured to be screwed on coupling interface 536 such as to secure shaft 502 to handle 504. More specifically, threaded cover 564 is not affixed onto shaft 502, in the sense of being moveable along shaft body 506 (when adaptor 524 and coupling interface 536 are not interlocked). For example, as depicted in FIGS. 5A and 5B, threaded cover 564 may include a hole 572 (on a cover top 574), whereby threaded cover 564 is mounted on shaft body 506, such as to allow shifting threaded cover 564 along shaft body 506. When threaded cover 564 is being screwed on coupling interface 536—due to shaft proximal tip member 554 having a greater diameter than the rest of shaft body 506—cover top 574 is pressed against shaft proximal tip member 554, so that not only is shaft 502 mechanically secured to handle 504 but shaft proximal tip member 554 is affixed as well, such as to electronically couple adaptor 524 to coupling interface 536.

According to some embodiments, adaptor 524 and coupling interface 536 include matching keyed components/patterns which dictate a preferred coupling orientation. For example, and as depicted in FIGS. 5A and 5B, adaptor 524 may include at least one tooth 582 proximally projecting from a rim 584 of shaft proximal tip member 554, and circumferential surface 562 may include at least one slot 586 (i.e. a notch or groove) extending proximally from handle distal end 558 and matching at least one tooth 582, respectively, such as to dictate the preferred coupling orientation. In particular, according to some embodiments (not depicted), when adaptor 524 includes a plurality of teeth and circumferential surface 562 includes a plurality of corresponding slots, then the teeth may be asymmetrically disposed on shaft proximal tip member 554 or differ from one another e.g. in size, such as to allow the coupling of adaptor 524 and coupling interface 536 only at a single (preferred) orientation.

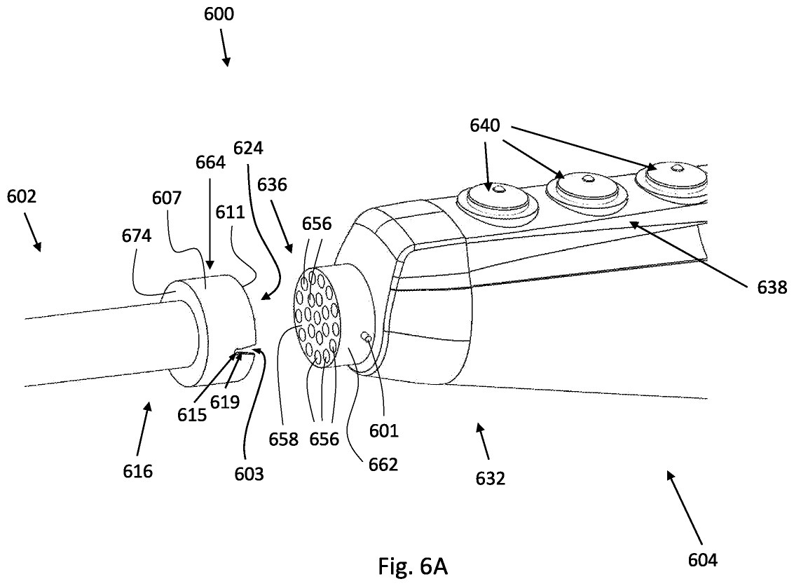

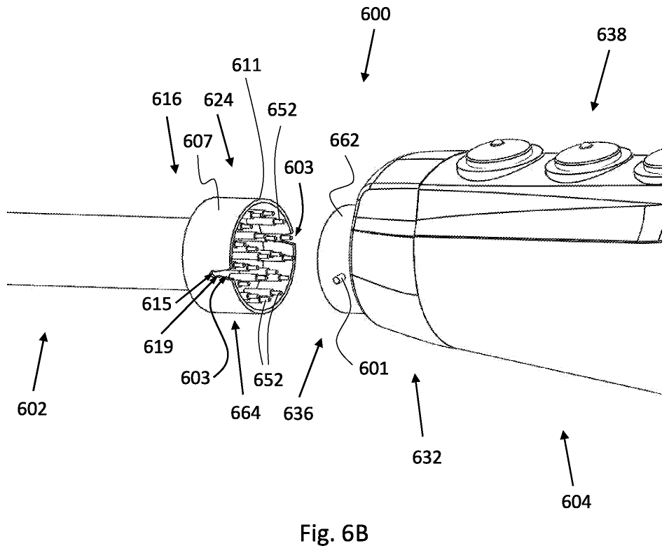

FIGS. 6A and 6B are schematic, perspective (partial) views of a rigid endoscope 600, according to some embodiments. Endoscope 600 is a specific embodiment of endoscope 100 and includes alternative interlocking mechanisms to those depicted in FIGS. 5A and 5B. Endoscope 600 includes an elongated shaft 602 and a handle 604, which are specific embodiments of elongated shaft 102 (or elongated shaft 402) and handle 104, respectively. More specifically, in FIGS. 6A and 6B a shaft proximal section 616 and a handle distal section 632 are depicted. Shaft proximal section 616 includes an adaptor 624 and handle distal section 632 includes a coupling interface 636. Adaptor 624 and coupling interface 636 are specific embodiments of adaptor 124 and coupling interface 136 and are configured to be electronically coupled and interlocked via a snap interlocking mechanism, as described below.

According to some embodiments, and as depicted in FIGS. 6A and 6B, adaptor 624 includes one or more spring-loaded pins 652 proximally projecting from an inner surface (not indicated) of a top 674 of a cover 664, which is positioned on shaft proximal section 616 (and forms part of adaptor 624). Coupling interface 636 may include one or more flat conductive surfaces 656, corresponding to the one or more spring-loaded pins 652, on a handle distal end 658. Nevertheless, it will be understood that other electronic coupling mechanisms are applicable, such as (non-spring loaded) pins and corresponding pinholes, as described hereinabove in the description of FIGS. 1A and 1B. Coupling interface 636 may be cylindrical, including a circumferential surface 662, and cover 664 may include a circumferential inner surface, such that cover 664 is configured to receive therein coupling interface 636, as described below.

According to some embodiments, and as depicted in FIGS. 6A and 6B, coupling interface 636 includes one or more radial pins 601, which radially project from circumferential surface 662, and cover 664 includes one or more snap-female receptors 603 on a circumferential surface 607 of cover 664. Each of snap-female receptors 603 (as a non-limiting example, two snap-female receptors are depicted in FIG. 6B) forms a slot (i.e. a notch) extending distally from a rim 611 of cover 664. Each of the of the slots is A-shaped, with the base of the “A” being positioned on rim 611 and a respective “head” 615 of the “A” being positioned distally relative to the base. Each of heads 615 is “mounted” on a respective “neck” 619 (of the “A”). Heads 615 may be circular and characterized by a diameter greater than a thickness of necks 619. According to some embodiments, the diameter of heads 615 is equal to or slightly greater than the diameter characterizing pins 601. When snap-female receptors 603 and pins 601 are interlocked, each of pins 601 is “gripped” by a respective head from heads 615. In particular, the thickness of necks 619 is smaller than the diameter of pins 601, such as to facilitate the snap-engagement between pins 601 and snap-female receptors 603.

To interlock (mechanically couple) coupling interface 636 and adaptor 624, coupling interface 636 and adaptor 624 must be oriented with respect to one another, such as to allow the insertion of each of pins 601 into a respective one of the slots on cover 664. According to some embodiments, snap-female receptors 603 may be asymmetrically disposed on cover 664, and, accordingly, pins 601 may be similarly asymmetrically disposed on circumferential surface 662, such as to allow the coupling of adaptor 624 and coupling interface 636 only at a single (preferred) orientation. Coupling interface 636 may then be proximally pushed until each of pins 601 is snap-engaged by the respective slot (i.e. the respective snap-female receptor from snap-female receptors 603). That is, until each of pins 601 is forced into the respective head from heads 615. Cover 664 is made of a material (or includes a material around snap-female receptors 603) that is sufficiently flexible or pliable to allow for pins 601 to be forced into heads 615 without necks 619 breaking or deforming in a manner such as to affect, or substantially affect, the strength/quality of the grip on pins 601 provided by heads 615.

Also indicated in FIGS. 6A and 6B, are a user control interface 638 including buttons 640, which are specific embodiment of user control interface 138 and buttons 140.

It will be understood that other designs of adaptor 624 and coupling interface 636, not depicted in FIGS. 6A and 6B, may apply: In particular, according to some embodiments, coupling interface 636 may include spring-loaded pins or pins and adaptor 624 may include corresponding flat conductive surfaces or pinholes, respectively. According to some embodiments, adaptor 624 does not include cover 664 and coupling interface 636 does not include pins 601, instead coupling interface 636 includes a cover similar to cover 664, and adaptor 624 includes pins similar to pins 601. In particular, according to some such embodiments, the cover (included in coupling interface 636) includes snap-female receptors similar to snap-female receptors 603.

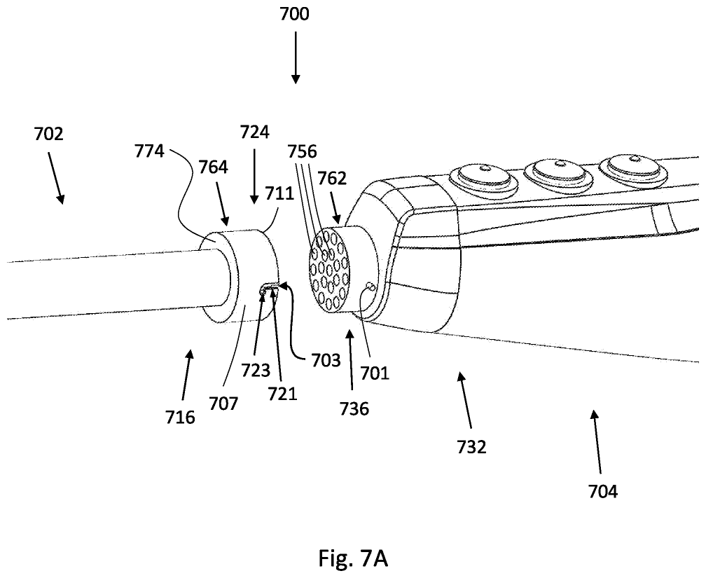

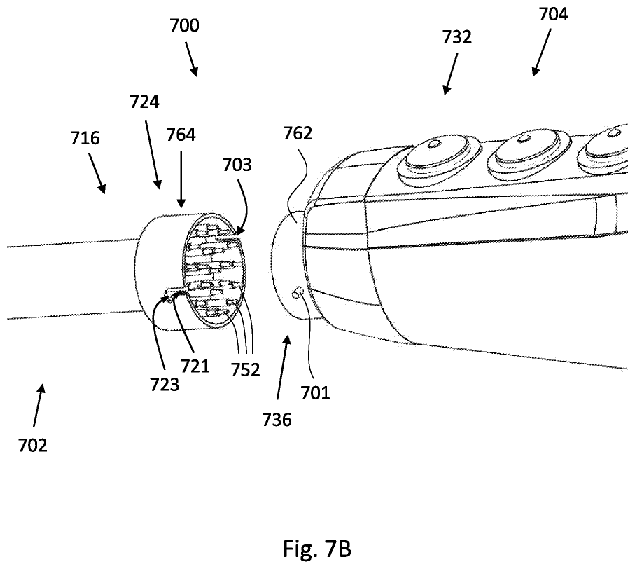

FIGS. 7A and 7B are schematic, perspective (partial) views of an endoscope 700, according to some embodiments. Endoscope 700 is a specific embodiment of endoscope 100 and includes alternative interlocking mechanisms to those depicted in FIGS. 5A and 5B and in FIGS. 6A and 6B. Endoscope 700 includes an elongated shaft 702 and a handle 704, which are specific embodiments of elongated shaft 102 (or elongated shaft 402) and handle 104, respectively. More specifically, in FIGS. 7A and 7B a shaft proximal section 716 and a handle distal section 732 are depicted. Shaft proximal section 716 includes an adaptor 724 and handle distal section 732 includes a coupling interface 736. Adaptor 724 and coupling interface 736 are specific embodiments of adaptor 124 and coupling interface 136 and are configured to be electronically coupled and interlocked via a bayonet connector interlocking mechanism, as described below.

Endoscope 700 is similar to endoscope 600 but differs therefrom in the interlocking mechanism. According to some embodiments, handle 704 may be essentially similar to handle 604, and elongated shaft 702 may differ from elongated shaft 602 in including one or more L-shaped slots, instead of A-shaped slots, but may otherwise be essentially similar thereto. More specifically, according to some embodiments, and as depicted in FIGS. 7A and 7B, adaptor 724 includes one or more spring-loaded pins 752 proximally projecting from an inner surface (not indicated) of a top 774 of a cover 764, which is positioned on shaft proximal section 716 and forms part of adaptor 724. Coupling interface 736 may include one or more flat conductive surfaces 756 corresponding to the one or more spring-loaded pins 752, on a handle distal end 758. Nevertheless, it will be understood that other electronic coupling mechanisms are applicable, such as (non-spring loaded) pins and corresponding pinholes, as described hereinabove in the description of FIGS. 1A and 1B. Coupling interface 736 may be cylindrical (i.e. including a circumferential surface 762), and cover 764 may include a circumferential inner surface, such that cover 764 is configured to receive therein coupling interface 736, as described below.

According to some embodiments, and as depicted in FIGS. 7A and 7B, coupling interface 736 includes one or more radial pins 701, which may be essentially similar to radial pins 601 of coupling interface 636, and cover 764 includes one or more snap-female receptors 703, respectively, on a circumferential surface 707 of cover 764. Each of snap-female receptors 703 (as a non-limiting example, two snap-female receptors are depicted in FIG. 7B) differs from each of snap-female receptors 603 in being L-shaped instead of A-shaped.

Each of the snap-female receptors 703 is positioned on cover 764 such that a vertical part 721 of the “L” extends distally from a rim 711 of cover 764 and terminates in a base 723 of the “L”, which extends along circumferential surface 707. In particular, all of bases 723 extend in the same sense (i.e. either clockwise or anti-clockwise) so as to facilitate the interlocking of adaptor 724 and coupling interface 736, as explained below. Further, each of bases 723 may include narrowed portion, such as to facilitate the snap-engagement and gripping of a respective pin from pins 701.

It is noted that while in FIGS. 7A and 7B the “L”-s are tilted sidewise, the vertical part of the “L” will nevertheless be understood to refer to the vertical line defined by the “L” when the “L” is positioned upright, as when represented in the alphabet and or when read in a text. Similarly, the base of the “L” will be understood to refer to the horizontal line from which the vertical part extends upwards when the “L” is positioned upright.

To interlock coupling interface 736 and adaptor 724, coupling interface 736 and adaptor 724 must be oriented with respect to one another such as to allow the insertion of each of pins 601 into a respective one of the slots on cover 764, i.e. to insert each of pins 601 into vertical part 721 of the respective slot. According to some embodiments, adaptor 724 and coupling interface 736 are configured to allow interlocking thereof only at a single (preferred) orientation. According to some such embodiments, snap-female receptors 703 may be asymmetrically disposed on cover 764, and, accordingly, pins 701 may be similarly asymmetrically disposed on circumferential surface 762, such that unless oriented with respect to one another at the single (preferred) orientation, adaptor 724 and coupling interface 736 cannot be coupled, as at least one of pins 701 will not be properly positioned relative to the respective snap-female receptor. Coupling interface 736 may then be proximally pushed until each of pins 701 reaches the distal end of the respective vertical part of the slot, following which coupling interface 736 may be turned (either clockwise or anticlockwise depending on the sense in which bases 723 all point) until each of pins 701 is snap-engaged by a respective base from bases 723. Cover 764 may be made of a material (or includes a material around snap-female receptors 703) that is sufficiently flexible or pliable such as to allow for pins 701 to be forced into bases 723 without any breaking or deformation of snap-female receptors 703 in a manner such as to affect, or substantially affect, the strength/quality of the grip on pins 701 provided by bases 723.

It will be understood that other designs of adaptor 724 and coupling interface 736, not depicted in FIGS. 7A and 7B, may apply: In particular, according to some embodiments, coupling interface 736 may include spring-loaded pins or pins and adaptor 724 may include corresponding flat conductive surfaces or pinholes, respectively. According to some embodiments, adaptor 724 does not include cover 764 and coupling interface 736 does not include pins 701, instead coupling interface 736 includes a cover similar to cover 764, and adaptor 724 includes pins similar to pins 701. In particular, according to some such embodiments, the cover (included in coupling interface) includes snap-female receptors similar to snap-female receptors 703.

It will be understood that the scope of the disclosure also covers shafts for semi-rigid endoscopes. As used herein, according to some embodiments, a “semi-rigid endoscope” may refer to an endoscope including a semi-rigid shaft. The semi-rigid shaft may include a rigid elongated member, a distal tip portion, and a maneuvering portion mounted between, and mechanically coupling, the elongated member and the distal tip portion. The semi-rigid shaft includes at least two cameras: a front camera and one or more side-cameras. The front camera is positioned on the distal tip portion. Each of the one or more side cameras may be positioned on the distal tip portion, the maneuvering portion, or the elongated member. The semi-rigid shaft further includes one or more illumination components configured to illuminate the FOV provided by the at least two cameras. The maneuvering portion is configured to bend, rotate, and/or angulate the distal tip portion, and thereby controllably change the combined FOV provided by the at least two cameras.

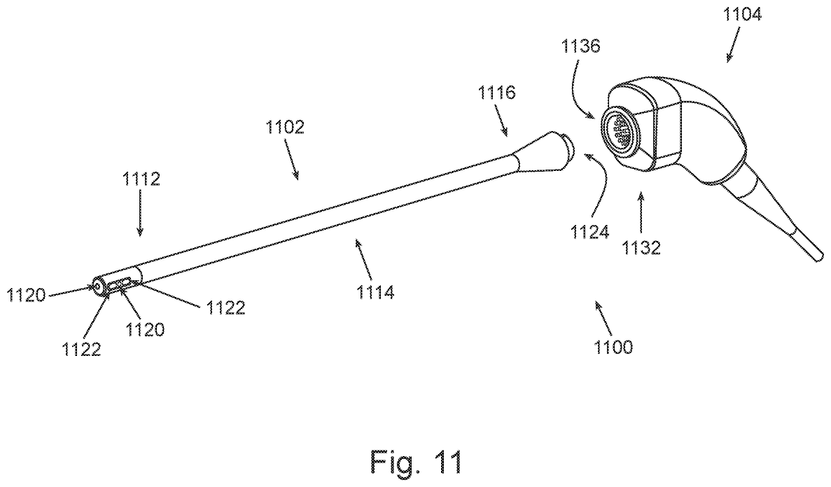

Thus, according to an aspect of some embodiments, not depicted in the figures, there is provided a semi-rigid endoscope. The semi-rigid endoscope may be similar to any one of endoscopes 100 (whether including shaft 102 or shaft 402), 500, 600, 700, and the endoscope depicted in FIG. 11, but differs therefrom in including a semi-rigid shaft, as described in the preceding paragraph, instead of a rigid shaft. In particular, the at least two cameras and illumination components of the semi-rigid endoscope may be similar to cameras 120 and illumination components 122.

According to an aspect of some embodiments, there is provided an endoscope including an elongated shaft mounted on a handle. The shaft includes a shaft distal section (which may also be referred to as “imaging component”), a shaft central section, and a shaft proximal section. The shaft distal section, which may be similar to shaft distal section 412 of elongated shaft 402, is detachable from the rest of the endoscope and may be discarded after a single use. The rest of the endoscope may be reusable. According to some embodiments, wherein the shaft is rigid, the shaft is similar to some embodiments of elongated shaft 402 but differs therefrom in that only the shaft distal section is detachable from the rest of the endoscope (i.e. the rest of the shaft is not detachable from the handle, unlike elongated shaft 402 which may be fully detached). According to some embodiments, the endoscope is semi-rigid.

According to some embodiments, each of endoscopes 100, 500, 600, and 700 may be (i) directly maneuvered by a user through the manipulation of handles 104, 504, 604, and 704, respectively, as well as (ii) indirectly maneuvered, via robotics, e.g. using a robotic arm or other suitable gripping means configured to allow manipulation of handles 104, 504, 604, and 704, respectively.

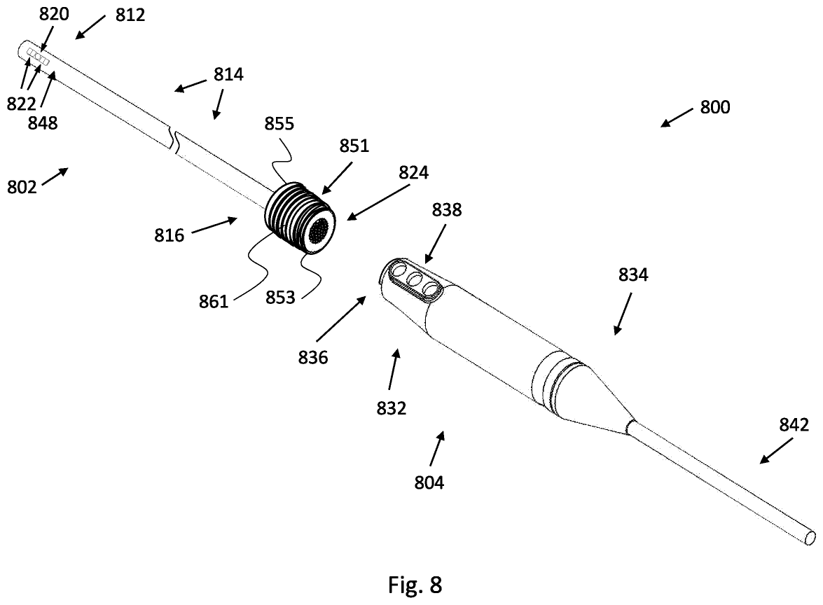

FIG. 8 is a schematic, perspective view of a rigid endoscope 800, according to some embodiments. Endoscope 800 includes an elongated shaft 802 and a handle 804, which are specific embodiments of elongated shaft 102 (or elongated shaft 402) and handle 104, elongated shaft 502 and handle 504, elongated shaft 602 and handle 604, or elongated shaft 702 and handle 704, respectively. Each possibility corresponds to separate embodiments. In FIG. 8, endoscope 800 is shown in a disassembled state, wherein shaft 802 is detached from handle 804.

Endoscope 800 further includes a sleeve 851, shown wrapped on shaft 802. Sleeve 851 is configured to be pulled over handle 804, such as to protect handle 804 from body fluids and debris during an endoscopy procedure (that is, prevent body fluids and debris from reaching handle 804), while affording a user a comfortable grip on handle 804 and comfortable usage of a user control interface 838 on handle 804, as explained below.

Shaft 802 includes a shaft distal section 812, a shaft central section 814, and a shaft proximal section 816. Shaft distal section 812 includes at least two cameras 820, only a side camera of which is shown in FIG. 8, and illumination components 822, with only illumination components on a first side-surface 848 of shaft distal section 812 being shown in FIG. 8. Handle 804 includes a handle distal section 832 and a handle proximal section 834. Shaft proximal section 816 includes an adaptor 824 and handle distal section 832 includes a coupling interface 836. Adaptor 824 and coupling interface 836 may be specific embodiments of adaptor 124 and coupling interface 136, adaptor 524 and coupling interface 536, adaptor 624 and coupling interface 636, or adaptor 724 and coupling interface 736, respectively, or similar thereto. Each possibility corresponds to separate embodiments. According to some embodiments, and as depicted in FIG. 8, endoscope 800 further includes, or is connectable to, a utility cable 842, configured for coupling endoscope 800 to a main control unit, such as main control unit 210, and/or an external power source.

Sleeve 851 includes a sleeve first end 853 and a sleeve second end 855. Sleeve first end 853 may be sealably attached to shaft proximal section 816, as elaborated on below. Sleeve second end 855 may be open. Sleeve 851 may be fluid-proof in the sense of (i) being made of a fluid-proof material and (ii) not including any perforations (in the sheet making up the sleeve). According to some embodiments, sleeve 851 may be made of, or include, a polymeric material, such as thermoplastic including nylon, polyurethane, polyamide, and/or the like. Sleeve first end 853 attachment to shaft proximal section 816 is fluid-tight in the sense of not leaving any space for passage of fluid (e.g. blood and other body fluids) or debris (e.g. bits of tissue) between sleeve first end 853 and shaft proximal section 816. Further, the attachment may include, or form a sealant, to prevent the passage of fluid and debris. According to some embodiments, sleeve first end 853 is sealably attached to shaft proximal section 816, for example, by bonding means such as glue, a snap connector(s), and/or the like. Additionally or alternatively, according to some embodiments, the sealable (fluid-tight) attachment of sleeve first end 853 to shaft proximal section 816 may be effected using heat-fusion, ultrasonic welding, and/or the like. According to some embodiments, sleeve first end 853 may be attached to a shaft proximal end 854 (i.e. the proximal end of shaft 802), or proximately thereto.

According to some embodiments, shaft 802 may be provided in an initial configuration wherein sleeve 851 is wrapped on shaft 816. FIG. 8 schematically depicts sleeve 851 in the initial configuration, according to some embodiments. As used herein, according to some embodiments, a sleeve, such as sleeve 851, may be said to be “wrapped” on a shaft (an elongated member, a rod), such as shaft 802, when rolled up, folded, or otherwise collected or gathered on the shaft. According to some embodiments, a sleeve may be said to be wrapped on a shaft when measuring no more than about a half, about a third, about, a fourth, about a fifth, or about a tenth of the length thereof when unwrapped and stretched. Each possibility corresponds to different embodiments.

According to some embodiments, shaft 802 and sleeve 851 are disposable, while handle 804 is reusable. According to some embodiments, shaft distal section 812 is detachable from the rest of shaft 802, and the rest of shaft 802 (i.e. shaft central section 814 and shaft proximal section 816) and sleeve 851 are disposable, while shaft distal section 812 and handle 804 are reusable. According to some embodiments, shaft 802, sleeve 851, and handle 804 are disposable, and shaft 802 and/or handle 804 are wirelessly communicatively associated with a main control unit, such as main control unit 210 of FIG. 2.

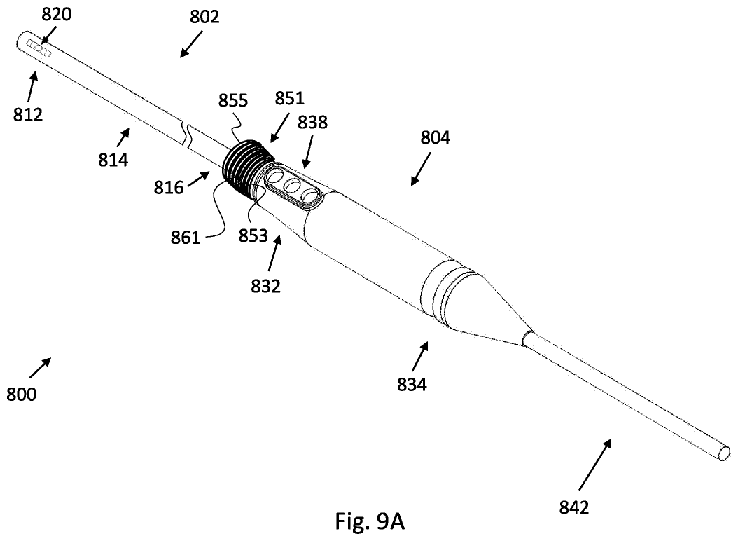

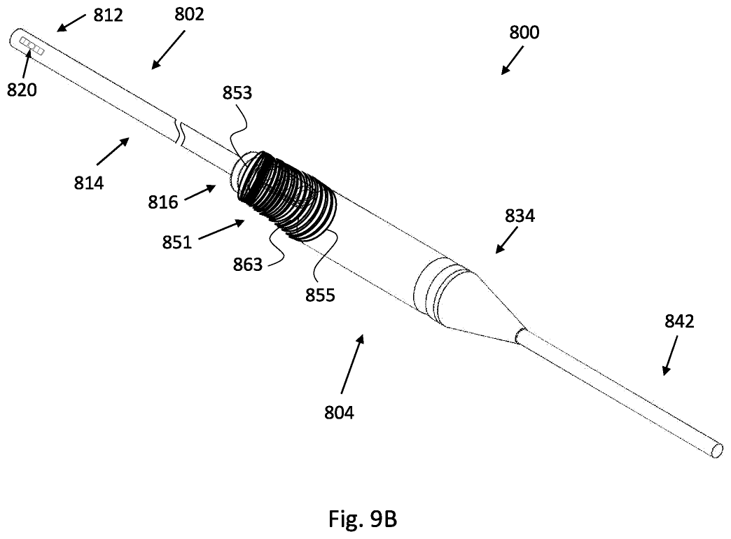

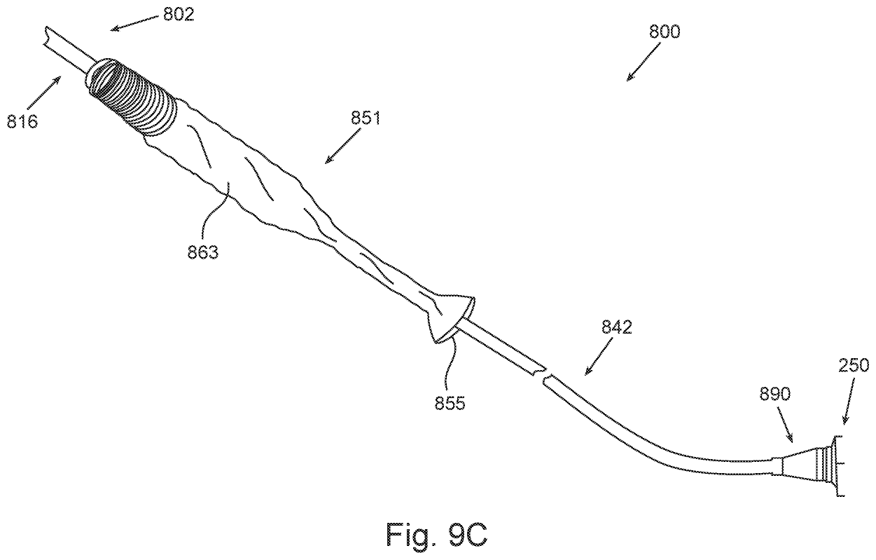

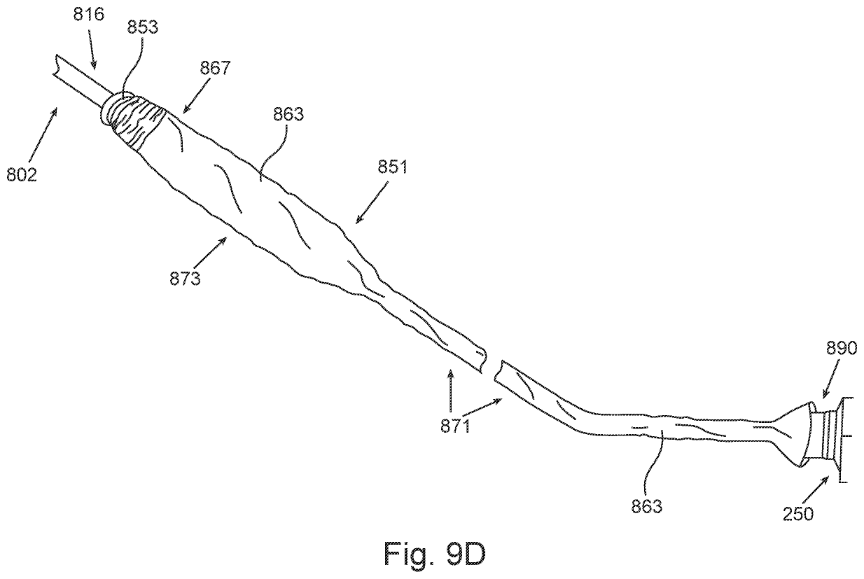

Making reference also to FIGS. 9A-9D, FIGS. 9A-9D schematically depict successive stages in the unwrapping of sleeve 851, and the pulling of sleeve 851 over handle 804 and cable 842, from an initial sleeve configuration (shown in FIG. 9A) wherein sleeve 851 is fully wrapped on shaft proximal section 816 to a final sleeve configuration (shown in FIG. 9D) wherein sleeve 851 is fully pulled over handle 804 and over cable 842, according to some embodiments. To begin unwrapping sleeve 851, sleeve second end 855 may be pulled in the proximal direction so that the internal and external surfaces of sleeve 851 are gradually inverted. That is, in FIG. 9A, a first surface 861 (shown also in FIG. 8) of sleeve 851 is visible, whereas a second surface 863 (shown in FIGS. 9B-9D) of sleeve 851 is not visible. In particular, in FIG. 9A sleeve second end 855 is positioned distally relative to sleeve first end 853. In FIGS. 9B-9D, second surface 863 is visible, while first surface 861 is not visible. In particular, in FIGS. 9B-9D sleeve 851 has been pulled “inside-out”, so that sleeve second end 855 is positioned proximally relative to sleeve first end 853. In FIG. 9B, sleeve 851 is shown collected on handle distal section 832. In FIG. 9C, sleeve 851 has been further unwrapped, as compared to in FIG. 9B, and is shown fully covering handle 804 and partially covering cable 842 (i.e. a distal segment of cable 842). In FIG. 9D, sleeve 851 has been fully unwrapped and is shown fully covering both handle 804 and cable 842, thereby preventing body fluids and debris from reaching handle 804 and cable 842, for example, when shaft 802 is inserted into an anatomical site during a medical procedure.

According to some embodiments, at least a sleeve portion 867 of sleeve 851—sleeve portion 867 including sleeve first end 853 or being positioned close thereto—may be transparent or semi-transparent so as to allow a user to clearly see user control interface 838 (which may be a specific embodiment of user control interface 138) on handle distal section 832 when sleeve 851 is fully unwrapped and pulled over handle 804.

According to some embodiments, sleeve second end 855 is configured to be further pulled, such as to cover a main connector 250 included in main control unit 210. Main connector 250 is adapted to receive a plug 890 or a port of cable 842, similar to plug 144 or a port of cable 142 of endoscope 100. According to some embodiments, sleeve second end 855 includes a connection means, such as a snap connector (not shown), configured to allow a user, when sleeve 851 is fully unwrapped, to easily anchor sleeve second end 855 to main connector 250 and to easily detach sleeve second end 855 therefrom (e.g. when the endoscopy procedure is finished).

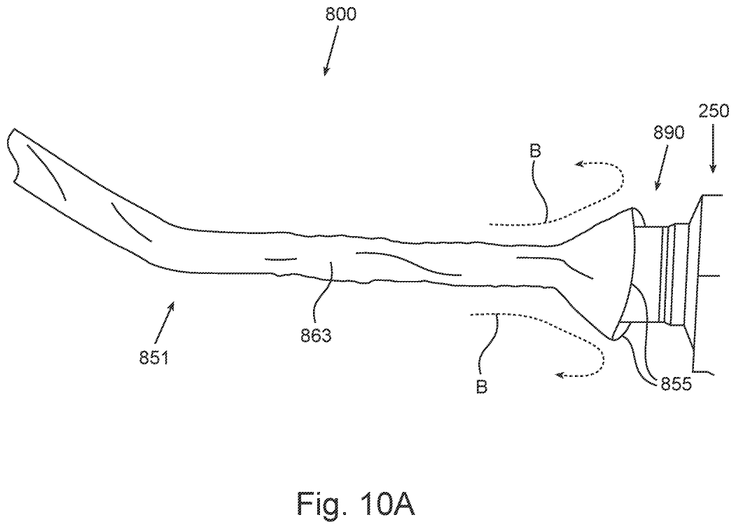

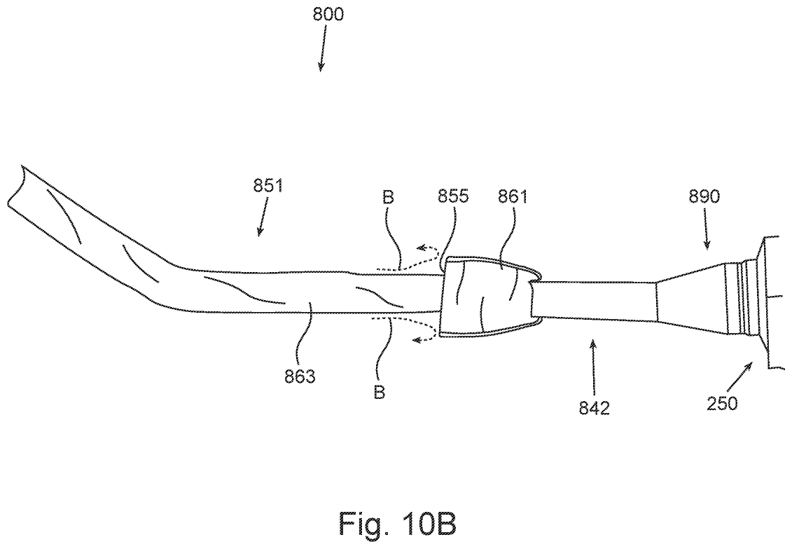

FIGS. 10A and 10B schematically depict removal of sleeve 851 from cable 842 (and handle 804 which is not shown). Making reference to FIG. 10A, starting from a state wherein sleeve 851 is fully pulled over handle 804 and cable 842 yet not connected to main connector 250, in order to begin the removal, sleeve second end 855 is pulled in the distal direction in a manner similar to a peeling motion, as indicated by arrows B. Making reference also to FIG. 10B, as sleeve second end 855 is pulled in the proximal direction, second surface 863 (which is fully visible in FIG. 10A) and first surface 861 (which is not visible in FIG. 10A but partially visible in FIG. 10B) start being inverted. When sleeve 851 has been fully pulled off of/withdrawn from cable 842 and handle 804, shaft 802 may be detached from handle 804 and discarded, in full, or in part, together with sleeve 851. For example, in embodiments wherein shaft distal section 812 is detachable, shaft distal section 812 may be kept while the rest of shaft 802 is discarded together with sleeve 851.

The shape of sleeve 851 may be adapted to the shape of endoscope 800, particularly so as to facilitate a comfortable grip of handle 804 by a user. In particular, a first portion 871 of sleeve 851, which is configured to extend over cable 842, typically when sleeve 851 is unwrapped and covers both handle 804 and cable 842, may be generally narrower than a second portion 873 (including sleeve portion 867) of sleeve 851, which is configured to extend over handle 804. It will be understood that if a sterile sleeve is incorporated in an endoscope, such as the endoscope depicted in FIG. 11, which is shaped differently from endoscope 800, the shape of the sterile sleeve may differ from that of sleeve 851 in order to conform to the shape of the endoscope to which the sterile sleeve is attached. Similarly, in embodiments wherein endoscope 800 is wireless and includes a replaceable and/or rechargeable battery, sleeve 851 will be understood to be accordingly shaped and dimensioned.

According to some embodiments, endoscope 800 may be (i) directly maneuvered by a user through the manipulation of handle 804, as well as (ii) indirectly maneuvered using robotics, in which case, handle 804 is gripped by a robotic arm or other suitable robotic gripping means.

It will be understood that the scope of the disclosure also covers semi-rigid endoscopes including a sterile sleeve. Thus, according to an aspect of some embodiments, there is provided a semi-rigid endoscope. The semi-rigid endoscope may be similar to endoscope 800 but differs therefrom at least in including a semi-rigid shaft instead of a rigid shaft. The semi-rigid shaft includes a rigid elongated member, a distal tip portion, and a maneuvering portion mounted between, and mechanically coupling, the elongated member and the distal tip portion. The semi-rigid shaft further includes a sleeve, such as sleeve 851 or a differently shaped sleeve (e.g. when the handle of the endoscope is shaped differently from handle 804). In an initial configuration, the sleeve may be wrapped on the elongated member in a similar manner to the wrapping of sleeve 851 on shaft 802 depicted in FIG. 8. According to some embodiments, the semi-rigid endoscope includes a plurality of cameras such as cameras 820. According to some other embodiments, the rigid endoscope includes a single camera.

FIG. 11 schematically depicts a rigid endoscope 1100, according to some embodiments. Endoscope 1100 may similar to each of endoscopes 100, 500, 600, and 700 but differs therefrom in the shape of the handle thereof. More specifically, endoscope 1100 includes an elongated shaft 1102 and a handle 1104. Shaft 1102 is detachable from handle 1104 essentially as described above in the descriptions of endoscopes 100, 500, 600, and 700. Elongated shaft 1102 may be essentially similar to any one of elongated shafts 102, 402, 502, 602, and 702. Handle 1104 differs from handles 104, 504, 604, and 704 in shape but is otherwise functionally similar thereto. In particular, handle 1104 may be shaped similarly to a pistol handle, so that endoscope 1100 resembles a pistol in shape, while endoscopes 100, 500, 600, and 700 may be essentially shaped as a linear rod (whose diameter may change along the length thereof).

Indicated in FIG. 11 are a shaft distal section 1112, a shaft central section 1114, and a shaft proximal section 1116. Also indicated are cameras 1120 and illumination components 1122 on shaft distal section 1112, an adaptor 1124 on shaft proximal section 1116, and a coupling interface 1136 on a handle distal section 1132.

According to some embodiments, endoscope 1100 may be (i) directly maneuvered by a user through the manipulation of handle 1104, as well as (ii) indirectly maneuvered using robotics, in which case, handle 1104 is gripped by a robotic arm or other suitable robotic gripping means.

According to some embodiments, not depicted in FIG. 11, endoscope 1100 includes a sterile sleeve similar to sterile sleeve 851 but differing therefrom in the shape and dimensions thereof such as to be adapted to endoscope 1100.

According to an aspect of some embodiments, there is provided a rigid endoscope, not depicted in the figures. The endoscope is similar to endoscope 800, in the sense of including a sleeve similar to sleeve 851, but differs from endoscope 800 in including only a single camera instead of two cameras or more.

Persons of ordinary skill in the art should appreciate that handles 804, 1104 may be maneuvered by a user performing a medical procedure either directly or throughout the maneuvering of robotics.

As used herein, according to some embodiments, “shaft” and “elongated shaft” are used interchangeably. Similarly, according to some embodiments, “shaft distal section” and “shaft body distal section” are interchangeable, “shaft central section” and “shaft body central section” are interchangeable, and “shaft proximal section” and “shaft body proximal section” are interchangeable.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1.-32. (canceled)

33. An elongated shaft detachably mountable on a reusable handle of a multi-camera endoscope, the elongated shaft comprising a shaft body, the shaft body comprising at a shaft body distal section at least two cameras and at least one illumination component, and, at a shaft body proximal section, an adaptor;
wherein the adaptor is configured to mechanically and electronically detachably couple to a coupling interface on a distal section of the handle, such as to mount the elongated shaft on the handle; and
wherein the adaptor is further configured to dictate a preferred mounting orientation such that the at least two cameras provide a combined and predetermined at least about 270 degrees horizontal field-of-view (FOV) of a target area within an anatomical cavity when the elongated shaft is mounted on the handle.

34. The elongated shaft of claim 33, wherein the elongated shaft further comprises a sterile sleeve wrapped on the shaft body, the sleeve comprising a sleeve first end, which is circumferentially attached to the shaft body proximal section, and a sleeve second end;
wherein the sleeve is configured to be proximally pulled over the handle when the elongated shaft is mounted on the handle, thereby unwrapping the sleeve; and
wherein the attachment between the sleeve first end and the shaft body proximal section is fluid-tight, thereby preventing fluids and debris from anatomical cavities, into which the elongated shaft is insertable, from reaching the handle when the sleeve is pulled over the handle.

35. The elongated shaft of claim 33, wherein the at least two cameras comprise a front camera on a distal tip of the shaft and a first side-camera.

36. The elongated shaft of claim 35, wherein the at least two cameras further comprise a second side-camera, wherein the first side-camera and the second side-camera are positioned on opposite sides of the shaft, and wherein the first side-camera is positioned distally relative to the second side-camera.

37. The elongated shaft of claim 33, wherein the elongated shaft is disposable.

38. The elongated shaft of claim 33, wherein the shaft body distal section is detachable.

39. The elongated shaft of claim 38, wherein the shaft body distal section is reusable and the rest of the of the elongated shaft is disposable.

40. The elongated shaft of claim 33, wherein the adaptor comprises an interlocking component, which is complementary to an interlocking component on the coupling interface, the interlocking components being configured to prevent coupling between the adaptor and the coupling interface except at the preferred orientation.

41. The elongated shaft of claim 40, wherein the adaptor comprises a keyed component, which is complementary to a keyed component on the coupling interface, the keyed components being configured to prevent coupling between the adaptor and the coupling interface except at the preferred orientation.

42. The elongated shaft of claim 40, wherein the interlocking component comprises one or more snap-female receptors, each configured to snap-engage a corresponding pin on the coupling interface.

43. The elongated shaft of claim 40, wherein the interlocking component comprises one or more pins, each configured to be snap-engaged by corresponding snap-female receptor in the coupling interface.

44. The elongated shaft of claim 33, wherein the adaptor comprises a spring-loaded pin component, which is configured to engage a corresponding flat conductive surface on the coupling interface when the adaptor is coupled to the coupling interface at the preferred orientation.

45. The elongated shaft of claim 35, wherein the adaptor comprises a flat conductive surface, which is configured to engage a spring-loaded pin component on the coupling interface when the adaptor is coupled to the coupling interface at the preferred orientation.

46. The elongated shaft of claim 33, wherein the shaft body is rigid or semi-rigid.

47. The elongated shaft of claim 33, wherein each of the at least one illumination component is or comprises discrete light source.

48. A multi-camera endoscope comprising an elongated shaft of claim 33, and a reusable handle, wherein the elongated shaft is detachably mountable on the handle.

49. The multi-camera endoscope of claim 48, wherein the elongated shaft and/or the handle comprise an authentication mechanism configured to prevent operation of the multi-camera endoscope unless the shaft is authenticated.

50. The multi-camera endoscope of claim 49, wherein a used shaft is inauthentic.

51. An imaging component detachably mountable on a distal end of an elongated member of a multi-camera endoscope, the imaging component comprising at least two cameras, at least one illumination component, and an adaptor electrically associated with the at least two cameras and the at least one illumination component;
wherein the adaptor is configured to mechanically and electronically detachably couple the imaging component to the distal end of the elongated member; and
wherein the adaptor is further configured to dictate a preferred coupling orientation, such that the at least two cameras provide a combined and predetermined horizontal field-of-view (FOV) of at least about 270 degrees of a target area within an anatomical cavity, when coupled to the elongated member.

52. The imaging component of claim 51, wherein the imaging component is disposable.
